(19) United States
(12) Patent Application Publication
(10) Pub. No.: US 2023/0149613 A1
(43) Pub. Date: May 18, 2023

(54) DEVICES AND METHODS FOR TREATING OR PREVENTING CYTOKINE RELEASE SYNDROME AND TUMOR LYSIS SYNDROME

(71) Applicant: SeaStar Medical, Inc., Denver, CO (US)

(72) Inventor: James R. Matson, Westminster, CO (US)

(21) Appl. No.: 18/002,576

(22) PCT Filed: Jun. 28, 2021
(86) PCT No.: PCT/US2021/039301

§ 371 Date: Dec. 20, 2022

Related U.S. Application Data
(60) Provisional application No. 63/045,503, filed on Jun. 29, 2020.

(51) Int. Cl. A61M 1/34; A61P 37/06; A61K 35/17

(52) U.S. Cl. CPC A61M 1/3401; A61P 37/06; A61K 35/17; A61M 2202/0445

(57) ABSTRACT

The invention relates generally to methods of treating or reducing the risk of Cytokine Release Syndrome (CRS), treating or reducing the risk of Tumor Lysis Syndrome (TLS), or increasing drug exposure in a subject undergoing CAR T-cell therapy or chemotherapy. The methods comprise contacting blood from the subject with an extracorporeal membrane having a plurality of pores having an average pore size of at least 60 kDa to permit inflammatory cytokines, other inflammatory molecules, or metabolites to pass through the pores and out of the blood that is returned back to the subject.

### CROSS-REFERENCE TO RELATED APPLICATIONS

This application claims priority to and the benefit of U.S. Provisional Patent Application No. 63/045,503, filed Jun. 29, 2020, the contents of which are incorporated by reference herein in their entirety.

### FIELD OF THE INVENTION

The invention involves treatment of blood by extracorporeal circulation in a subject with or at risk for Cytokine Release Syndrome (CRS) or Tumor Lysis Syndrome (TLS), for example, a subject undergoing CAR T-cell therapy or chemotherapy.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner.

Chimeric antigen receptors (CARs) are synthetic receptors that retarget immune cells, e.g., T-cells, to tumor surface antigens (Sadelain et al. (2003), NAT. REV. CANCER. 3(1):35-45, Sadelain et al. (2013) CANCER DISCOVERY 3(4):388-398). CARs provide both antigen binding and immune cell activation functions. Initially, CARs contained an antibody-based tumor-binding element, such as a single chain Fv (scFv), that is responsible for antigen recognition linked to either CD3zeta or Fc receptor signaling domains, which trigger T-cell activation. Later CAR constructs included additional activating and costimulatory signaling domains. Recently, the CAR T-cell therapy KYMRIAH® (Tisagenlecleucel) received FDA approval for treatment of certain B-cell acute lymphoblastic leukemias (ALL) and large B-cell lymphomas and the CAR T-cell therapy YESCARTA® (Axicabtagene ciloleucel) received FDA approval for treatment of certain large B-cell lymphomas.

Cytokine release syndrome (CRS) is a potentially life-threatening, systemic inflammatory response that can be triggered by a variety of factors, including administration of certain drugs. CRS is one of the most frequent serious side effects of CAR T-cell therapies and other T-cell engaging immunotherapeutic agents (e.g., bispecific antibody constructs). Another common toxicity from CAR T-cell therapy (and related therapies) is Tumor lysis syndrome (TLS). TLS is characterized by massive tumor cell death leading to the development of metabolic complications and target organ dysfunction. TLS is most common in hematologic malignancies, many of which are very sensitive to anti-oncotic treatment responding with massive cell death and disruption causing spillage of intracellular contents i.e., potassium, phosphorus, and uric acid into the lymph and blood circulation. TLS may also occur spontaneously. Uric acid is directly toxic to kidneys; phosphorus load drives down serum calcium promoting cardiac arrythmias, which are also caused or aggravated by high plasma potassium. Death may result from cardiac or central nervous system toxic effects, or kidney failure. (Mirrakhimov et al., World J of Crit Care Med, 2015, 4:130-138).

Accordingly, there is a need for methods that treat or reduce the risk of CRS or TLS, including, for example, in subjects receiving CAR T-cell therapy or chemotherapy.

### SUMMARY OF THE INVENTION

The invention is based, in part, upon the discovery that outcomes in a subject with or at risk for Cytokine Release Syndrome (CRS) or Tumor Lysis Syndrome (TLS), for example, a subject undergoing CAR T-cell therapy or chemotherapy, can be improved by contacting blood from the subject with a semi-permeable membrane, for example, one or more membranes disposed in a hemofilter, whereupon, inflammatory cytokines or other inflammatory molecules can pass through semi-permeable membrane. Blood depleted of inflammatory molecules is then returned to the subject. Without wishing to be bound by theory, it is contemplated that processing the subject's blood using this approach removes inflammatory cytokines and other inflammatory molecules from the blood that cause inflammatory responses that ultimately lead to CRS and/or TLS.

Accordingly, in one aspect, the invention provides a method of reducing the risk of CRS in a subject undergoing CAR T-cell therapy or chemotherapy. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules in the blood to pass therethrough for removal from the blood. The blood depleted of inflammatory molecules is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules from the blood pass through the pores and are removed from the blood. The blood depleted of inflammatory molecules is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of increasing exposure to CAR T-cell therapy or chemotherapy in a subject undergoing CAR T-cell therapy or chemotherapy while minimizing the risk that the subject experiences CRS. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules in the blood to pass therethrough for removal from the blood. The blood depleted of inflammatory molecules is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules from the blood pass through the pores and are removed from the blood. The blood depleted of inflammatory molecules is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of reducing the risk of TLS in a subject undergoing CAR T-cell therapy or chemotherapy. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of increasing exposure to CAR T-cell therapy or chemotherapy in a subject undergoing CAR T-cell therapy or chemotherapy while minimizing the risk that the subject experiences TLS. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of treating TLS or CRS in a subject undergoing CAR T-cell therapy or chemotherapy. In one embodiment, the method treats TLS. In another embodiment, the method treats CRS. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. In one embodiment, the average pore size is at least 60 kDa. In one embodiment, the average pore size is at least 50 kDa. In one embodiment, the average pore size is at least 40 kDa.

In yet another aspect, the invention provides a method of removing inflammatory molecules from the blood of a subject experiencing CRS or TLS using an extracorporeal membrane defining a plurality of pores having an average pore size of at least 40 kDa, 50 kDa or 60 kDa to permit inflammatory molecules in the blood to pass through the pores for removal from the blood. The blood depleted of the inflammatory molecules is returned to the recipient. In one embodiment, the average pore size is at least 60 kDa. In one embodiment, the average pore size is at least 50 kDa. In one embodiment, the average pore size is at least 40 kDa.

In yet another aspect, the invention provides a method of removing inflammatory molecules from the blood of a subject suffering from CRS or TLS using an extracorporeal cartridge comprising a housing and a plurality of semi-permeable hollow fibers defining a lumen and a plurality of pores, such that when the blood to traverses the lumens of the hollow fibers, inflammatory molecules from the blood pass through the pores and are removed from the blood. The blood depleted of the inflammatory molecules is returned to the recipient.

In another aspect, the invention provides a method of treating cancer in a subject while reducing the risk that the subject experiences CRS or TLS comprising administering CAR T-cell therapy or chemotherapy to the subject. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. The dose of CAR T-cell therapy or of chemotherapy administered to the subject is greater than the dose that could be administered without contacting blood from the subject with an extracorporeal membrane. In another embodiment, the method involves passing blood from the subject through an extracorporeal cartridge comprising a housing and a plurality of semi-permeable hollow fibers disposed therein. For example, the semi-permeable hollow fibers are substantially parallel semi-permeable hollow fibers. Each of the semi-permeable hollow fibers comprises a lumen and a plurality of pores having an average pore size of at least 40 kDa, 50 kDa, or 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. The dose of CAR T-cell therapy or of chemotherapy administered to the subject is greater than the dose that could be administered without passing blood from the subject through the extracorporeal cartridge. In one embodiment, the average pore size is at least 60 kDa. In one embodiment, the average pore size is at least 50 kDa. In one embodiment, the average pore size is at least 40 kDa.

In certain embodiments of any of the foregoing methods, the metabolites resulting from tumor cell lysis are one or more of uric acid, potassium and phosphorus. For example, in certain embodiments, (i) the method maintains the level of uric acid in the subject at or below 476 μmol/L, (ii) the method maintains the level of potassium in the subject at or below 6 mmol/L, and/or (iii) the method maintains the level of phosphorus in the subject at or below 1.45 mmol/L.

In certain embodiments of any of the foregoing methods, the chemotherapy is an antibody therapy, e.g., a bispecific antibody therapy. In certain embodiments, the chemotherapy is a non-protein based chemotherapeutic. In certain embodiments, the chemotherapeutic is anti-thymocyte globulin (ATG), TGN1412, rituximab, obinutuzumab, alemtuzumab, brentuximab, dacetuzumab, nivolumab, oxaliplatin, lenalidomide, or blinatolimumab.

In certain embodiments of any of the foregoing methods, the CAR T-cell therapy is KYMRIAH® (Tisagenlecleucel) or YESCARTA® (Axicabtagene ciloleucel). In certain embodiments, the CAR T-cell therapy is administered at a dose greater than the U.S. Food and Drug Administration approved dose. For example, in certain embodiments, the CAR T-cell therapy is KYMRIAH® and the dose of KYMRIAH® is (i) greater than 5.0×10⁶ CAR-positive viable T cells per kg weight intravenously and the subject is a pediatric or young adult subject up to 25 years of age with B-cell acute lymphoblastic leukemia weighing 50 kg or less, (ii) greater than 2.5×10⁸ CAR-positive viable T cells intravenously and the subject is a pediatric or young adult subject with B-cell acute lymphoblastic leukemia weighing 50 kg or more, or (iii) greater than 6.0×10⁸ CAR-positive viable T cells and the subject is an adult subject with relapsed or refractory diffuse large B-cell lymphoma. In certain embodiments, the CAR T-cell therapy is YESCARTA® and the dose of YESCARTA is (i) greater than 2×10⁶ CAR-positive viable T cells per kg body weight, or (ii) greater than 2×10⁸ CAR-positive viable T cells.

In certain embodiments of any of the foregoing methods, the subject has a hematologic malignancy, for example, acute lymphoblastic B cell leukemia (B-ALL), chronic lymphocytic leukemia (CLL), diffuse large B cell lymphoma (DLBCL), acute myeloid leukemia (AML), or Burkitt Lymphoma.

In certain embodiments of any of the foregoing methods, the method is performed on the subject within 0-24 hours or within 0-72 hours after the subject receives a dose of the chemotherapy or CAR T-cell therapy. In certain embodiments, the method is performed on the subject prior to the subject exhibiting any symptoms of CRS.

In certain embodiments of any of the foregoing methods, the subject also has a reduced risk of developing Cytokine Release Syndrome (CRS) as a result of the method. In certain embodiments, the method reduces the likelihood the subject will experience sepsis from concomitant infection with a bacteria, virus or fungus. In certain embodiments, the method prevents the need for dose-reduction of the chemotherapy or CAR T-cell therapy to prevent CRS. In certain embodiments, the method negates the need for the subject to receive allopurinol or a phosphate binder. In certain embodiments, the method negates the need for the subject to receive corticosteroids.

In certain embodiments of any of the foregoing methods, the subject does not receive tocilizumab prior to or subsequent to administration of a dose of chemotherapy or CAR T-cell therapy. In certain embodiments, the subject receives exchange of recombinant or pharmaceutical grade albumin.

In certain embodiments of any of the foregoing methods, the pores have an average pore size of from about 60 kDa to about 150 kDa. In other embodiments, the average pore size is greater than 65 kDa. In other embodiments, the average pore size is no greater than 65 kDa. In still further embodiments, the average pore size is from about 60 kDa to about 65 kDa. In certain embodiments, the pores are defined by a wall (e.g., an inner wall or an outer wall) of a semi-permeable hollow fiber. In some embodiments, the average pore size is about 40 kDa or greater. In some embodiments, the average pore size is about 50 kDa or greater. In other embodiments, the average pore size is no greater than 40 kDa. In other embodiments, the average pore size is no greater than 50 kDa. In other embodiments, the pores have an average pore size of from about 40 kDa to about 150 kDa. In other embodiments, the pores have an average pore size of from about 50 kDa to about 150 kDa. In one embodiment, the average pore size is at least 60 kDa.

In still further embodiments, the molecular weight cut-off the membrane or the hollow fibers is about 65 kDa, where, for example, molecules greater than 65 kDa do not readily pass through the pores of the membrane or hollow fibers. In other embodiments, molecules less than 65 kDa can pass through the pores of the membrane or hollow fibers.

In certain embodiments of any of the foregoing methods, the subject is a human, e.g., an adult human or a pediatric human.

In certain embodiments of any of the foregoing methods, the inflammatory molecules removed from the blood are selected from one or more of IL-4, IL-6, IL-8, TNF-α, IL-1β, MCP-1, CCL2, IP-10, CXCL10, C3a, C5a, soluble TNF receptor II, soluble TNF receptor I, matrix metalloproteinase-9, matrix metalloproteinase-7, IL-10, soluble gp130, lipopolysaccharide (LPS), or procalcitonin. In certain embodiments, the inflammatory molecules removed from the blood are selected from one or more of IL-6, TNF-α, C3a, and C5a.

In certain embodiments of any of the foregoing methods, the membrane or the hollow fibers comprise a polymer. In certain embodiments, the polymer is polysulfone. In certain embodiments, the lumen of the hollow fibers has a diameter of from about 100 μM to about 700 μM. In certain embodiments, the diameter is from about 175 μM to about 225 μM. In another embodiment, the diameter is from about 600 μM to about 700 μM. In certain embodiments, the surface area of the hollow fibers is from about 0.01 m² to about 4.0 m², e.g., from about 1.9 m² to about 2.1 m², from about 0.05 m²to about 0.1 m², from about 0.25 m² to about 0.75 m², or from about 1.0 m²to 1.5 m².

In certain embodiments of any of the foregoing methods, the cartridge comprises a fluid inlet port and a fluid outlet port and/or the cartridge comprises one or more ultrafiltrate ports.

In certain embodiments of any of the foregoing methods, the flow rate of blood through the cartridge is from about 100 mL/min to about 600 mL/min. For example, in some embodiments, the flow rate is from about 100 mL/min to about 400 mL/min, from about 150 mL/min to about 300 mL/min, or from about 150 mL/min to about 250 mL/min.

In certain embodiments of any of the foregoing methods, the subject's blood is passed through the cartridge or contacted with the membrane more than once, for example, 1, 2, 3, 4, 5, 6, 7, 8, 9, 10, 11, 12, 14, 16, 18, 20, 22, 24, or more than 24 times. In certain embodiments, the subject's blood is passed through the cartridge or contacted with the membrane multiples times (e.g., repeatedly, e.g., at regular intervals) over a treatment period. For example, in certain embodiments, the subject's blood is passed through the cartridge or contacted with the membrane for about 1 hour, about 2 hours, about 3 hours, about 4 hours, about 5 hours, about 6 hours, about 9 hours, or about 12 hours, about every 3 hours, about every 6 hours, about every 12 hours, about every day, about every 24 hours, about every 2 days, about every 3 days, about every 4 days, about every 5 days, about every 6 days or about every 7 days, over a period of about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks.

In certain embodiments of any of the foregoing methods, the cartridge or membrane is connected to the subject via an extracorporeal circuit or extracorporeal circulation system comprising a line from an artery of the subject and a line to a vein of the subject. In certain embodiments, the extracorporeal circuit or circulation system further comprises an ultrafiltrate collection line and/or container. In certain embodiments, the cartridge or membrane is connected to the subject via an extracorporeal circuit or extracorporeal circulation system comprising a line from a vein of the subject and a line to a vein of the subject. In certain embodiments, the extracorporeal circuit or circulation system further comprises an ultrafiltrate collection line and/or container. In certain embodiments, the extracorporeal circuit or circulation system comprises a double lumen catheter inserted in to a vein of the subject enabling pumping of blood from the vein and returning of blood to the vein. In certain embodiments, the extracorporeal circuit or circulation system further comprises one or more of an ultrafiltrate pump, ultrafiltrate pressure sensor, blood sensor, filter pressure sensor, venous pressure sensor, access pressure sensor, IV fluid return pump, ultrafiltration controller, or a temperature regulator.

In certain embodiments of any of the foregoing methods, the ultrafiltration rate of the cartridge is from about 1 mL/min to about 180 mL/min, e.g., from about 40 mL/min to about 180 mL/min.

These and other aspects and features of the invention are described in the following detailed description and claims.

### DESCRIPTION OF THE DRAWINGS

The invention can be more completely understood with reference to the following drawings, in which

FIG. 1 is a schematic representation of an exemplary cartridge;

FIG. 2 is a schematic representation of an exemplary extracorporeal blood circuit used for a subject;

FIG. 3 is a schematic representation of an exemplary extracorporeal blood circuit used for a subject; and

FIG. 4 is a schematic representation of an exemplary extracorporeal blood circuit used for a subject.

### DETAILED DESCRIPTION

The invention is based, in part, upon the discovery that outcomes in a subject with or at risk for Cytokine Release Syndrome (CRS) and/or Tumor Lysis Syndrome (TLS), for example, a subject undergoing CAR T-cell therapy or chemotherapy, can be improved by contacting blood from the subject with a semi-permeable membrane, for example, one or more membranes disposed in a hemofilter, whereupon, inflammatory cytokines or other inflammatory molecules can pass through semi-permeable membrane. Blood depleted of inflammatory molecules is then returned to the subject. Without wishing to be bound by theory, it is contemplated that processing the subject blood using this approach removes inflammatory cytokines and other inflammatory molecules from the blood that cause inflammatory responses that ultimately lead to CRS and/or TLS.

Accordingly, in one aspect, the invention provides a method of reducing the risk of CRS in a subject undergoing CAR T-cell therapy or chemotherapy. In one embodiment, the method involves contacting blood from the subject with an extracorporeal membrane. The extracorporeal membrane defines a plurality of pores having an average pore size of at least 40 kDa, 50 kDa or 60 kDa that permit inflammatory molecules in the blood to pass therethrough for removal from the blood. The blood depleted of inflammatory molecules is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores in the walls of the hollow fibers such that when the blood traverses the lumens of the hollow fibers, inflammatory molecules from the blood pass through the pores and are removed from the blood. The blood depleted of inflammatory molecules is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of increasing exposure to CAR T-cell therapy or chemotherapy in a subject undergoing CAR T-cell therapy or chemotherapy while minimizing the risk that the subject experiences CRS. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules in the blood to pass therethrough for removal from the blood. The blood depleted of inflammatory molecules is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules from the blood pass through the pores and are removed from the blood. The blood depleted of inflammatory molecules is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of reducing the risk of TLS in a subject undergoing CAR T-cell therapy or chemotherapy. In one embodiment, the method involves contacting blood from the subject with an extracorporeal membrane. The extracorporeal membrane defines a plurality of pores having an average pore size of at least 40 kDa, 50 kDa, or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. In one embodiment, the average pore size is at least 60 kDa. In one embodiment, the average pore size is at least 50 kDa. In one embodiment, the average pore size is at least 40 kDa.

In another aspect, the invention provides a method of increasing drug exposure in a subject undergoing CAR T-cell therapy or chemotherapy while minimizing the risk that the subject experiences TLS. In one embodiment, the method involves contacting blood from the subject with an extracorporeal membrane. The extracorporeal membrane defines a plurality of pores having an average pore size of at least 40 kDa, 50 kDa or 60 kDa that permit inflammatory molecules and metabolites resulting from tumor lysis in the blood to pass therethrough for removal from the blood. The blood depleted of inflammatory molecules and metabolites resulting from tumor lysis is then returned to the subject. In another embodiment, the method involves passing blood from the subject through an extracorporeal cartridge comprising a housing and a plurality of semi-permeable hollow fibers disposed therein. Each of the semi-permeable hollow fibers comprises a lumen and a plurality of pores having an average pore size of at least 60 kDa in the walls of the hollow fibers such that when the blood traverses the lumens of the hollow fibers, inflammatory molecules and metabolites resulting from tumor lysis in the blood pass through the pores and are removed from the blood. The blood depleted of inflammatory molecules and metabolites is then returned to the subject. In one embodiment, the average pore size is at least 60 kDa. In one embodiment, the average pore size is at least 50 kDa. In one embodiment, the average pore size is at least 40 kDa.

Various features and aspects of the invention are discussed in more detail below.

### I. Membranes/Cartridges

The methods of the invention relate to contacting blood from a subject with an extracorporeal membrane. The membrane includes a plurality of pores having an average pore size of at least 40 kDa, 50 kDa, or 60 kDa that, for example, permit inflammatory molecules to be removed from the blood so that blood depleted of inflammatory molecules can be returned to the subject. In one embodiment, the average pore size is at least 60 kDa. In one embodiment, the average pore size is at least 50 kDa. In one embodiment, the average pore size is at least 40 kDa.

In certain embodiments, the extracorporeal membrane is disposed in a cartridge. Although the underlying principles for designing an appropriate cartridge are discussed in detail, it is understood that cartridges useful in the practice of the invention are not limited to the particular design configurations discussed herein. In certain embodiments, a cartridge useful in the practice of the invention may, for example, comprise a housing and a plurality of semi-permeable hollow fibers disposed therein, each of the semi-permeable hollow fibers comprising a lumen and a plurality of pores. The cartridge may further comprise a fluid inlet port and a fluid outlet port and/or one or more ultrafiltrate ports.

Other cartridges useful in the practice of the invention include one or more fluid permeable membranes capable of filtering inflammatory molecules from the blood.

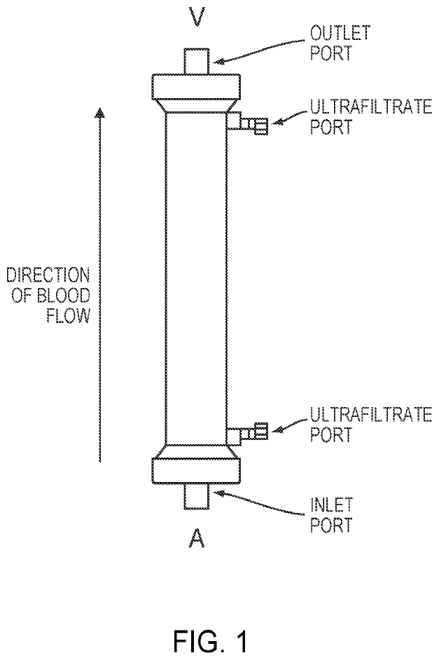

For example, as shown in FIG. 1, blood may enter the cartridge through a fluid inlet port (e.g., an arterial inlet port), pass through the hollow fibers and exit at the opposite end through a fluid outlet port (e.g., a venous outlet port).

It is understood that the membrane or hollow fibers in the cartridge used for filtration are not limited to a particular type, kind or size, and may be made of any appropriate material; however, the material should be biocompatible. For example, a surface of the membrane or fibers may be any biocompatible polymer comprising one or more of nylon, polyethylene, polyurethane, polyethylene terephthalate (PET), polytetrafluoroethylene (PTFE), CUPROPHAN (a cellulose regenerated by means of the cuprammonium process, available from Enka), HEMOPHAN (a modified CUPROPHAN with improved biocompatibility, available from Enka), CUPRAMMONIUM RAYON (a variety of CUPROPHAN, available from Asahi), BIOMEMBRANE (cuprammonium rayon available from Asahi), saponified cellulose acetate (such as fibers available from Teijin or CD Medical), cellulose acetate (such as fibers available from Toyobo Nipro), cellulose (such as that are regenerated by the modified cuprammonium process or by means of the viscose process, available from Terumo or Textikombinat (Pirna, Germany) respectively), polyacrylonitrile (PAN), polysulfone, polyethersulfone, polyarylethersulfone, acrylic copolymers (such as acrylonitrile-NA-methallyl-sulfonate copolymer, available from Hospal), polycarbonate copolymer (such as GAMBRONE, a fiber available from Gambro), polymethylmethacrylate copolymers (such as fibers available from Toray), ethylene vinyl copolymer (such as EVAL, an ethylene-vinyl alcohol copolymer available from Kuraray), polyvinylalcohol, polyamide, and polycarbonate. Alternatively, a surface may be nylon mesh, cotton mesh, or woven fiber. The surface can have a constant thickness or an irregular thickness. In some embodiments, fibers may include silicon, for example, silicon nanofabricated membranes (see, e.g., U.S. Patent Publication No. 2004/0124147). In certain embodiments, the membrane or hollow fibers in the cartridge used for filtration include a polysulfone, e.g., glycerin-free polysulfone. Other suitable biocompatible fibers are known in the art, for example, in Salem and Mujais (1993) DIALYSIS THERAPY 2D ED., Ch. 5: Dialyzers, Eds. Nissensen and Fine, Hanley & Belfus, Inc., Philadelphia, Pa.

Depending upon the subject and the application, the surface area of the membrane or hollow fibers in the cartridge used for filtration may be from about 0.01 m² to about 4.0 m². For example, the surface area of the hollow fibers may be from about 0.01 m² to about 3.0 m², about 0.01 m² to about 2.0 m², about 0.01 m² to about 1.0 m², about 0.01 m² to about 0.5 m², about 0.01 m² to about 0.1 m², about 0.01 m² to about 0.05 m², about 0.05 m² to about 4.0 m², about 0.05 m² to about 3.0 m², about 0.05 m² to about 2.0 m², about 0.05 m² to about 1.0 m², about 0.05 m² to about 0.5 m², about 0.05 m² to about 0.1 m², about 0.1 m² to about 4.0 m², about 0.1 m² to about 3.0 m², about 0.1 m² to about 2.0 m², about 0.1 m² to about 1.0 m², about 0.1 m² to about 0.5 m², about 0.2 m² to about 0.4 m², about 0.25 m² to about 0.35 m², about 0.5 m² to about 4.0 m², about 0.5 m² to about 3.0 m², about 0.5 m² to about 2.0 m², about 0.5 m² to about 1.0 m², about 0.5 m² to about 0.75 m², about 1.0 m² to about 4.0 m², about 1.0 m² to about 3.0 m², about 1.0 m² to about 2.0 m², about 1.0 m² to about 1.5 m², about 1.75 m² to about 2.5 m², about 1.75 m² to about 2.25 m², about 2.0 m² to about 4.0 m², about 2.0 m² to about 3.0 m², or about 3.0 m² to about 4.0 m². In certain embodiments, the surface area of the hollow fibers is from about 1.9 m² to about 2.1 m², from about 0.05 m² to about 0.1 m², from about 0.25 m² to about 0.75 m², or from about 1.0 m² to 1.5 m². In certain embodiments, the surface area is about 2.0 m². It will be appreciated that the surface area will vary depending on the age and size of the subject. For example, pediatric and infant subjects will require cartridges with smaller surface areas as compared to adult subjects; smaller adults may also require cartridges with smaller surface areas as compared to larger adults.

The surface area of the membrane or hollow fibers can be adapted by lengthening or shortening the length of the membrane or fibers. For example, the length of the membrane or hollow fibers may be about 30 cm, about 29 cm, about 28 cm, about 27 cm, about 26 cm, about 25 cm, about 24 cm, about 23 cm, about 22 cm, about 21 cm, about 20 cm, about 19 cm, about 18 cm, about 17 cm, about 16 cm, about 15 cm, about 14 cm, about 13 cm, about 12 cm, about 11 cm, about 10 cm, or about 5 cm.

The surface area of the hollow fibers can also be adapted by varying the number of hollow fibers used in the cartridge. In certain embodiments, the cartridge comprises from about 9,000 to about 15,000 hollow fibers. For example, the cartridge may comprise from about 9,000 to about 14,000, from about 9,000 to about 13,000, from about 9,000 to about 12,000, from about 9,000 to about 11,000, from about 9,000 to about 10,000, from about 10,000 to about 15,000, from about 10,000 to about 14,000, from about 10,000 to about 13,000, from about 10,000 to about 12,000, from about 10,000 to about 11,000, from about 11,000 to about 15,000, from about 11,000 to about 14,000, from about 11,000 to about 13,000, from about 11,000 to about 12,000, from about 12,000 to about 15,000, from about 12,000 to about 14,000, from about 12,000 to about 13,000, from about 12,000 to about 15,000, from about 12,000 to about 14,000, or from about 14,000 to about 13,000 hollow fibers.

Also depending upon the subject and the application, the lumen of hollow fibers in the cartridge may be from about 100 μM to about 700 μM. For example, the lumen may be from about 100 μM to about 700 μM, about 100 μM to about 600 μM, about 100 μM to about 500 μM, about 100 μM to about 400 μM, about 100 μM to about 300 μM, about 100 μM to about 200 μM, about 200 μM to about 700 μM, about 200 μM to about 600 μM, about 200 μM to about 500 μM, about 200 μM to about 400 μM, about 200 μM to about 300 μM, about 300 μM to about 700 μM, about 300 μM to about 600 μM, about 300 μM to about 500 μM, about 300 μM to about 400 μM, about 400 μM to about 700 μM, about 400 μM to about 600 μM, about 400 μM to about 500 μM, about 500 μM to about 700 μM, about 500 μM to about 600 μM, or about 600 μM to about 700 μM. In certain embodiments, the lumen of the hollow fibers has a diameter of about 175 μM to about 225 μM, or about 600 μM to about 700 μM. In certain embodiments, the lumen of the hollow fibers has a diameter of about 200 μM.

In certain embodiments, the hollow fibers are made of a semi-permeable membrane. The term “membrane” refers to a surface capable of receiving a fluid on both sides of the surface, or a fluid on one side and gas on the other side of the surface. It is understood that the sieving characteristics of a membrane depend not only on the pore size, but also on the physical, chemical, and electrical characteristics of the material from which the fiber or membrane is made, the particular manufacturing technique used and post production processing (e.g., sterilization). Nonetheless, the size of a pore in a porous membrane or fiber can be represented by a molecular weight cutoff (MWC or MWCO), i.e., the lowest molecular weight of solute in which 90% of the solute is retained by the membrane or fiber. Molecular weight cutoff may be measured by any method known in the art, including, for example, exposing the membrane or fiber to a solute with a known molecular weight (e.g., a polyethylene glycol or dextran) and ascertaining retention of the solute by the membrane or fiber. It is understood that the molecular weight cutoff may vary depending upon the conditions in which it is measured, for example, the molecular weight cutoff of a membrane or fiber that is measured when the membrane or fiber is disposed in an extracorporeal circuit including subject blood (i.e. the effective molecular weight cutoff) may be lower than the molecular weight cutoff of the membrane or fiber that is measured in a test situation (i.e. the nominal effective molecular weight cutoff). Further, flow rates, temperature, fluid type, and duration of use can impact the measurement of the molecular weight cutoff of a given membrane or fiber.

In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject blood. In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject plasma. In certain embodiments, a molecular weight cutoff or pore size of a membrane or fiber refers to the molecular weight cutoff or pore size of the membrane or fiber that is measured when the membrane or fiber is disposed in a circuit including water (e.g., aqueous saline). In certain embodiments, a molecular weight cutoff or pore size of a membrane or fiber refers to the molecular weight cutoff or pore size of the membrane or fiber that is measured when the membrane or fiber is disposed in a circuit (e.g., an extracorporeal circuit) including subject blood or plasma, and does not refer to the molecular weight cutoff or pore size of the membrane or fiber that is measured when the membrane or fiber is disposed in a circuit including water (e.g., aqueous saline).

A membrane or fiber can be porous (e.g., selectively porous or semi-porous) such that it is capable of fluid or gas flow therethrough. It is understood that the term “porous” as used herein to describe a surface, fiber, or membrane includes generally porous, selectively porous and/or semi-porous surfaces or membranes. A semi-permeable membrane refers to a membrane that permits only certain molecules to pass through while being impermeable to other molecules. In one embodiment, a membrane is semi-permeable based on the size of molecules contacting the membrane. For example, in one embodiment, a semi-permeable membrane is permeable to molecules below a certain size threshold while molecules above that size threshold are excluded from passing through the membrane.

Accordingly, in certain embodiments, the semi-permeable membrane or hollow fibers in the cartridge used for filtration of subject blood comprise a plurality of pores with an average pore size of from about 60 kDa to about 150 kDa. For example, the plurality of pores may have an average pore size of about 65 kDa to about 150 kDa, about 70 kDa to about 150 kDa, about 80 kDa to about 150 kDa, about 90 kDa to about 150 kDa, about 100 kDa to about 150 kDa, about 110 kDa to about 150 kDa, about 120 kDa to about 150 kDa, about 130 kDa to about 150 kDa, about 140 kDa to about 150 kDa, about 60 kDa to about 140 kDa, about 65 kDa to about 140 kDa, about 70 kDa to about 140 kDa, about 80 kDa to about 140 kDa, about 90 kDa to about 140 kDa, about 100 kDa to about 140 kDa, about 110 kDa to about 140 kDa, about 120 kDa to about 140 kDa, about 130 kDa to about 140 kDa, about 60 kDa to about 130 kDa, about 65 kDa to about 130 kDa, about 70 kDa to about 130 kDa, about 80 kDa to about 130 kDa, about 90 kDa to about 130 kDa, about 100 kDa to about 130 kDa, about 110 kDa to about 130 kDa, about 120 kDa to about 130 kDa, about 60 kDa to about 120 kDa, about 65 kDa to about 120 kDa, about 70 kDa to about 120 kDa, about 80 kDa to about 120 kDa, about 90 kDa to about 120 kDa, about 100 kDa to about 120 kDa, about 110 kDa to about 120 kDa, about 60 kDa to about 110 kDa, about 65 kDa to about 110 kDa, about 70 kDa to about 110 kDa, about 80 kDa to about 110 kDa, about 90 kDa to about 110 kDa, about 100 kDa to about 110 kDa, about 60 kDa to about 100 kDa, about 65 kDa to about 100 kDa, about 70 kDa to about 100 kDa, about 80 kDa to about 100 kDa, about 90 kDa to about 100 kDa, about 60 kDa to about 90 kDa, about 65 kDa to about 90 kDa, about 70 kDa to about 90 kDa, about 80 kDa to about 90 kDa, about 60 kDa to about 80 kDa, about 65 kDa to about 80 kDa, about 70 kDa to about 80 kDa, about 60 kDa to about 70 kDa, about 65 kDa to about 70 kDa, or about 60 kDa to about 65 kDa. The plurality of pores may have an average pore size greater than 60 kDa. The plurality of pores may have an average pore size greater than 65 kDa. The plurality of pores may have an average pore size greater than 70 kDa. The plurality of pores may have an average pore size greater than 80 kDa, greater than 90 kDa, greater than 100 kDa, greater than 110 kDa, greater than 120 kDa, greater than 130 kDa, greater than 140 kDa, or greater than 150 kDa. The plurality of pores may have an average pore size no greater than 65 kDa. The plurality of pores may have an average pore size from about 60 kDa to about 65 kDa. The plurality of pores may have an average pore size from about 40 kDa to 150 kDa. The plurality of pores may have an average pore size from about 50 kDa to 150 kDa. The plurality of pores may have an average pore size of about 40 kDa or greater. The plurality of pores may have an average pore size of about 50 kDa or greater. The plurality of pores may have an average pore size of not greater than 40 kDa. The plurality of pores may have an average pore size of not greater than 50 kDa.

In certain embodiments, the molecular weight cut-off the membrane or hollow fibers is about 65 kDa, where, for example, molecules greater than 65 kDa do not readily pass through the pores of the membrane or hollow fibers. In other embodiments, molecules less than 65 kDa can pass through the pores of the membrane or hollow fibers.

As used herein, the sieving coefficient (SC) of a membrane or fiber for a given solute refers to the ratio between the solute concentration in the filtrate and its concentration in the feed (e.g., blood, plasma, plasma water, or water). An SC of 1 indicates unrestricted transport while an SC of 0 indicates no transport at all. SC is specific for each fiber or membrane for each solute. It is understood that SC varies depending upon the treatment conditions, and measurement of the SC may even vary during treatment because the characteristics of the fiber or membrane may change.

In certain embodiments, the membrane or hollow fibers in the cartridge used for filtration have a sieving coefficient for IL-6 of about 0.8 to about 1.0. For example, the sieving coefficient for IL-6 may be from about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for IL-6 is at least 0.1, 0.2, 0.3, 0.4, 0.5, 0.6, 0.7, 0.8, 0.9, or 1.0. In certain embodiments, the sieving coefficient for IL-6 is about 1.0. In certain embodiments, the sieving coefficient for IL-6 is about 0.9. In certain embodiments, the sieving coefficient for IL-6 is greater than or equal to 0.1. In certain embodiments, the sieving coefficient for IL-6 may be measured as described in Clar et al. (1997) ASAIO J., 43:163-170.

In certain embodiments, the membrane or hollow fibers in the cartridge used for filtration have a sieving coefficient for urea of about 0.8 to about 1.0. For example, the sieving coefficient for urea may be from about 0.8 to about 1.0, from about 0.8 to about 0.9, or from about 0.9 to about 1.0. In certain embodiments, the sieving coefficient for urea is at least 0.8, 0.9, or 1.0. In certain embodiments, the sieving coefficient for urea is about 1.0. In certain embodiments, the sieving coefficient for urea may be measured in aqueous solution at a flow rate of 200 mL/min and transmembrane pressure (TMP) of 50 mmHg.

In certain embodiments, the membrane or hollow fibers in the cartridge used for filtration have a sieving coefficient for creatinine of about 0.8 to about 1.0. For example, the sieving coefficient for creatinine may be from about 0.8 to about 1.0, from about 0.8 to about 0.9, or from about 0.9 to about 1.0. In certain embodiments, the sieving coefficient for creatinine is at least 0.8, 0.9, or 1.0. In certain embodiments, the sieving coefficient for creatinine is about 1.0. In certain embodiments, the sieving coefficient for creatinine may be measured in aqueous solution at a flow rate of 200 mL/min and transmembrane pressure (TMP) of 50 mmHg.

In certain embodiments, the membrane or hollow fibers in the cartridge used for filtration have a sieving coefficient for vitamin B12 of about 0.8 to about 1.0. For example, the sieving coefficient for vitamin B12 may be from about 0.8 to about 1.0, from about 0.8 to about 0.9, or from about 0.9 to about 1.0. In certain embodiments, the sieving coefficient for creatinine vitamin B12 is at least 0.8, 0.9, or 1.0. In certain embodiments, the sieving coefficient for vitamin B12 is about 1.0. In certain embodiments, the sieving coefficient for vitamin B12 may be measured in aqueous solution at a flow rate of 200 mL/min and transmembrane pressure (TMP) of 50 mmHg.

In certain embodiments, the membrane or hollow fibers in the cartridge used for filtration have a sieving coefficient for myoglobin of about 0.10 to about 1.0. For example, the sieving coefficient for myoglobin may be from about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for myoglobin is at least 0.1, 0.2, 0.3, 0.4, 0.5, 0.6, 0.7, 0.8, 0.9, or 1.0. In certain embodiments, the sieving coefficient for myoglobin is about 1.0. In certain embodiments, the sieving coefficient for myoglobin is about 0.9. In certain embodiments, the sieving coefficient for myoglobin is greater than or equal to 0.1. In certain embodiments, the sieving coefficient for myoglobin is greater than or equal to 0.3 or is about 0.3 to about 0.4. . In certain embodiments, the sieving coefficient for myoglobin is at least 0.10, 0.15, 0.20, or 0.25. In certain embodiments, the sieving coefficient for myoglobin is about 0.17. In certain embodiments, the sieving coefficient for myoglobin is about 0.1 to about 0.25. In certain embodiments, the sieving coefficient for myoglobin may be measured in bovine blood at a flow rate of 400 mL/min and transmembrane pressure (TMP) of 400 mmHg.

In certain embodiments, the membrane or hollow fibers in the cartridge used for filtration have a sieving coefficient for albumin of about 0.005 to about 0.025. For example, the sieving coefficient for albumin may be from about 0.005 to about 0.025, about 0.005 to about 0.020, about 0.005 to about 0.015, about 0.005 to about 0.010, about 0.010 to about 0.025, about 0.010 to about 0.020, about 0.010 to about 0.015, about 0.015 to about 0.025, about 0.015 to about 0.020, or about 0.020 to about 0.025. In certain embodiments, the sieving coefficient for albumin is at least 0.005, 0.010, 0.015, 0.020, or 0.025. In certain embodiments, the sieving coefficient for albumin is about 0.015. In certain embodiments, the sieving coefficient for albumin is less than 0.1. In certain embodiments, the membrane or hollow fibers in the cartridge used for filtration have a sieving coefficient for albumin of about 0.2 to about 1.0. For example, the sieving coefficient for albumin may be from about 0.2 to about 1.0, about 0.2 to about 0.8, about 0.2 to about 0.6, about 0.2 to about 0.4, about 0.4 to about 1.0, about 0.4 to about 0.8, about 0.4 to about 0.6, about 0.6 to about 1.0, about 0.6 to about 0.8, or about 0.8 to about 1.0. In certain embodiments, the sieving coefficient for albumin is about 0.1 to about 1.0, about 0.1 to about 0.9, about 0.1 to about 0.8, about 0.1 to about 0.7, about 0.1 to about 0.6, about 0.1 to about 0.5, about 0.1 to about 0.4, about 0.1 to about 0.3, about 0.1 to about 0.2, about 0.2 to about 1.0, about 0.2 to about 0.9, about 0.2 to about 0.8, about 0.2 to about 0.7, about 0.2 to about 0.6, about 0.2 to about 0.5, about 0.2 to about 0.4, about 0.2 to about 0.3, about 0.3 to about 1.0, about 0.3 to about 0.9, about 0.3 to about 0.8, about 0.3 to about 0.7, about 0.3 to about 0.6, about 0.3 to about 0.5, about 0.3 to about 0.4, about 0.4 to about 1.0, about 0.4 to about 0.9, about 0.4 to about 0.8, about 0.4 to about 0.7, about 0.4 to about 0.6, about 0.4 to about 0.5, about 0.5 to about 1.0, about 0.5 to about 0.9, about 0.5 to about 0.8, about 0.5 to about 0.7, about 0.5 to about 0.6, about 0.6 to about 1.0, about 0.6 to about 0.9, about 0.6 to about 0.8, about 0.6 to about 0.7, about 0.7 to about 1.0, about 0.7 to about 0.9, about 0.7 to about 0.8, about 0.8 to about 1.4, about 0.8 to about 1.0, about 0.8 to about 0.9, or about 0.9 to about 1.0. In certain embodiments, the sieving coefficient for albumin may be measured in bovine blood at a flow rate of 400 mL/min and transmembrane pressure (TMP) of 400 mmHg.

In methods where the sieving coefficient of albumin exceeds 0.1, the organ recipient may receive albumin exchange intravenously to compensate for albumin loss. For example, the albumin may be recombinant albumin or ultrapure albumin. The recombinant albumin may be human or of another mammal. The ultrapure albumin may be mammalian ultrapure albumin, e.g., from a pig, horse, or cow. Recombinant human albumin suitable for exchange therapy is available from InVitria (Aurora, Colo.), AkronBiotech (Boca Raton, Fla.), and Valley Biomedical Products & Services, Inc. (Winchester, Va.). It is believed that recombinant or ultrapure albumin exchange may enhance the methods of the invention as the antioxidant potential of such albumin is enhanced compared to albumin sourced from human or mammalian plasma and therefore is able to bind more inflammatory mediators, cytokines, and toxins, removing them from the subject's blood.

The housing of the cartridge is not limited to a particular set of dimensions (e.g., length, width, weight, or another dimension). It is understood that the size and shape of the housing of the cartridge may be designed to provide the appropriate fill volume and to minimize turbulence when a fluid is passed through the cartridge. Furthermore, it is understood that the size, shape and composition of the membrane located within the cartridge may be designed to provide the appropriate surface area and to minimize turbulence when a fluid is passed through the cartridge. Also, the size of the cartridge depends upon the size of the subjects. For example, pediatric and infant subjects will require smaller cartridges areas as compared to adult subjects; smaller adults may also require smaller cartridges as compared to larger adults.

The housing of the cartridge can be fabricated from a variety of materials, but the material that defines that fluid contacting surface in the inner volume should be biocompatible. The cartridge can be constructed from a variety of materials including, metals such as titanium, or stainless steel with or without surface coatings of refractory metals including titanium, tantalum, or niobium; ceramics such as alumina, silica, or zirconia; polymers, such as polyvinylchloride, polyethylene, or polycarbonate; or plastic.

It is understood that the cartridge, once fabricated, should be sterilized prior to use. Sterility can be achieved through exposure to one or more sterilizing agents, separately or in combination, such as high temperature, high pressure, radiation, or chemical agents such as ethylene oxide, for example. The cartridge preferably is sterilized once it has been packaged, for example, after it has been hermetically sealed within an appropriate container (i.e., the cartridge is terminally sterilized). The sterilization process preferably achieves a sterility assurance level (SAL) of 10⁻³ or less; i.e., the probability of any given unit being nonsterile after the process is no more than 1 in 10³. More preferably, the sterilization process achieves an SAL of no more than 10⁻⁴, no more than 10⁻⁵, or no more than 10⁻⁶.

In certain embodiments, an extracorporeal cartridge useful in the methods described herein is the CLR 2.0 filter (SeaStar Medical, Inc., Denver, Colo.). In certain embodiments, cartridge (e.g., a CLR 2.0 cartridge) comprises semipermeable polysulfone hollow fibers that have a molecular weight cut-off of about 65 kDa.

### II. Blood Circuits

It is understood that a membrane or cartridge can be used in a variety of different fluid circuits or extracorporeal circulation system depending upon the intended use.

In basic form, an exemplary circuit includes a cartridge, a fluid connection for blood to flow from a blood source (for example, an artery or vein in a subject) to the cartridge, and a fluid connection for treated blood to flow from the cartridge to a receptacle (for example, back to a vein in the subject).

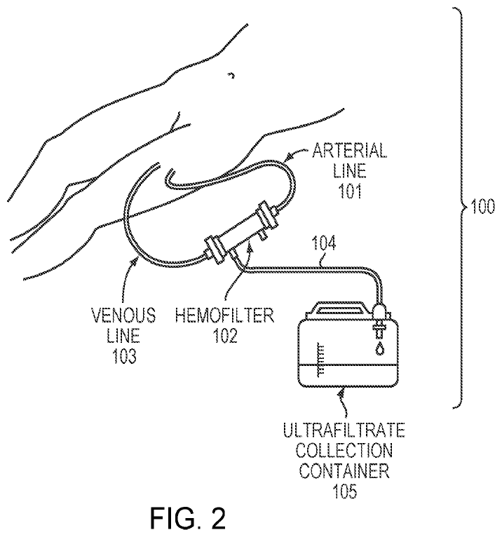

For example, as shown in the exemplary circuit 100 depicted in FIG. 2, blood flows from an artery in a subject to a blood line 101. Blood then enters the cartridge 102, passes through the hollow fibers (which run parallel to the length of the cartridge), and exits at the opposite end. Blood then flows through a blood line 103 and is returned to a vein in the subject. Hemofiltration occurs while the blood passes through the cartridge 102, with ultrafiltrate leaving the cartridge and passing through a line 104 to, for example, an ultrafiltrate collection container 105. During hemofiltration, plasma water and non-protein bound plasma solutes, including inflammatory molecules, are removed from the blood by ultrafiltration, while cellular elements and larger proteins are returned to the subject. For an exemplary cartridge, ultrafiltration occurs as a result of pressure gradients across the porous hollow fiber membrane. This gradient is achieved by: (1) positive pressure in the blood compartment, provided by the subject's mean arterial blood pressure and (2) negative pressure in the filtrate compartment, provided by the modest siphoning effect generated when ultrafiltrate is collected from the hemofilter (for example, in a container placed below the hemofilter). Replacement or substitution fluid may additionally be infused into the circuit 100 at a controlled rate to maintain fluid, electrolyte, acid base and nitrogen balance.

In certain embodiments, the extracorporeal circulation system further comprises one or more of an ultrafiltrate pump, ultrafiltrate pressure sensor, blood sensor, filter pressure sensor, venous pressure sensor, access pressure sensor, IV fluid return pump, ultrafiltration controller, or a temperature regulator.

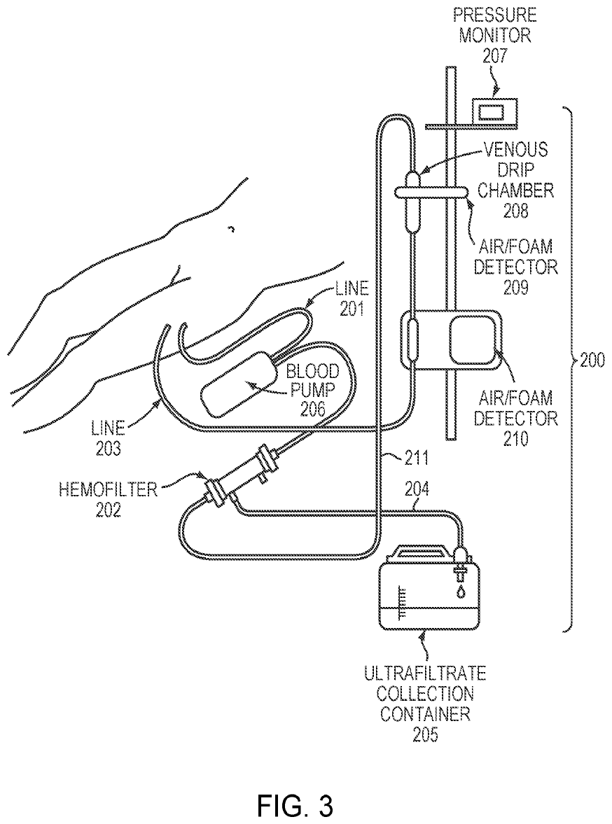

For example, in certain embodiments, a minimum flow rate is required for proper operation of the cartridge, and therefore one or more pumps may be necessary in subjects with systolic blood pressures below a certain threshold. For example, a pump assisted circuit 200 is shown in FIG. 3. Blood flows from an artery or vein in a subject to a blood line 201. Blood enters a pump 206 before continuing to the cartridge 202. Flow rates at the pump 206 can be chosen at ranges described herein. Blood enters the cartridge 202, passes through the hollow fibers, and exits at the opposite end. Hemofiltration occurs while the blood passes through the cartridge, with ultrafiltrate leaving the cartridge and passing through a line 204 to, for example, an ultrafiltrate collection container 205. Blood then flows through a blood line 211 and, prior to returning to a vein in the subject, passes through a venous drip chamber 208. The system may also include a pressure monitor 209 and air/foam detector 220.

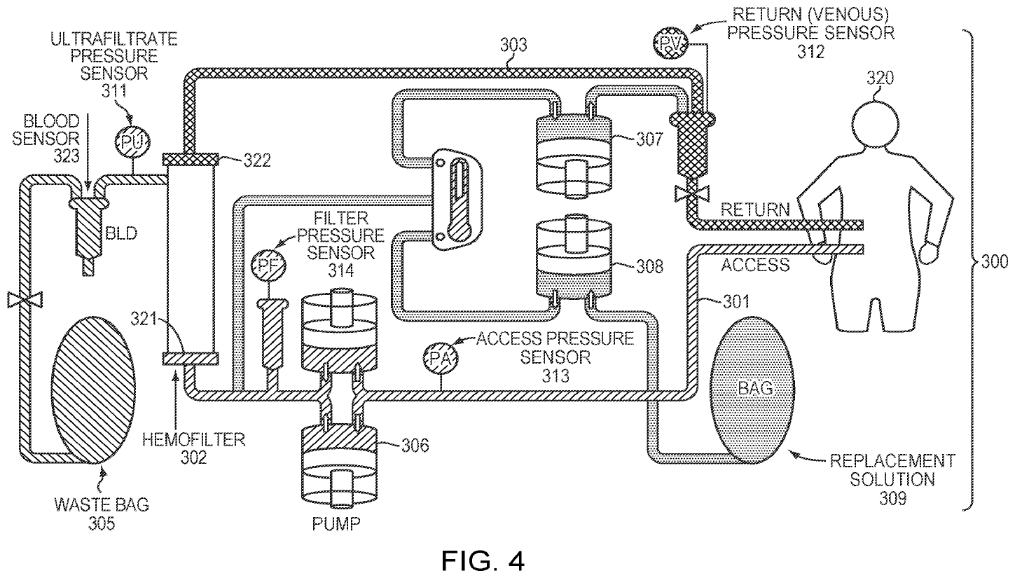

An additional exemplary circuit 300 is shown in FIG. 4. Blood flows from a subject 300 to a blood line 301. Blood enters a pump 306 before continuing to the cartridge 302. Flow rates at the pump 306 can be chosen at ranges described herein. Pressure is sampled at an access pressure sensor 313 prior to the pump, and a filter pressure sensor 314 after the pump and prior to the cartridge 302. Blood enters the cartridge 302 via a fluid inlet port 321, passes through the hollow fibers, and exits at the opposite end via a fluid outlet port 322. Hemofiltration occurs while the blood passes through the cartridge 302, with ultrafiltrate leaving the cartridge and passing through an ultrafiltrate line 304 to, for example, a waste bag 305. The ultrafiltrate line 304 may include an ultrafiltrate pressure sensor 311 and/or a blood sensor 323. Filtered blood leaves the cartridge 302 and flows through a blood line 303 to the subject. Pressure may be sampled at a return pressure sensor 312 prior to the returning to the subject. The system may also include a source of replacement solution. Replacement solution flows from a bag 309 to a pump 308, and is introduced into the circuit immediately prior to the cartridge and/or immediately prior to the return of fluid to the subject. Replacement solution temperature is monitored by a temperature regulator 310.

A cartridge may be connected to the subject's vascular system via vascular access which may include: arteriovenous femoral catheters, arteriovenous jugular catheters, Quinton-Scribner Shunt, arteriovenous fistula, veno-venous femoral catheters, veno-venous jugular catheters, veno-venous subclavian catheters, or veno-venous catheters at other sites. In certain embodiments, this is accomplished with a percutaneous catheter arrangement or an arteriovenous shunt. In certain embodiments, the extracorporeal circulation system comprises a double lumen catheter inserted in to a vein enabling pumping of blood from the vein and returning of blood to the vein.

The rate of blood flowing through the system will depend on the condition of the subject, the molecular weight cutoff of the associated fibers, the body size of subject, and other requirements for effective treatment of subjects. The amount of blood, the blood flow rate and the duration of treatment are preferably determined on a case by case basis after factoring the weight, the age and the nature of the subject.

In certain embodiments, the blood flow rate through the cartridge is from about 100 mL/min to about 600 mL/min. For example the blood flow rate may be from about 200 mL/min to about 600 mL/min, about 300 mL/min to about 600 mL/min, about 400 mL/min to about 600 mL/min, about 500 mL/min to about 600 mL/min, about 100 mL/min to about 500 mL/min, about 200 mL/min to about 500 mL/min, about 300 mL/min to about 500 mL/min, about 400 mL/min to about 500 mL/min, about 100 mL/min to about 400 mL/min, about 200 mL/min to about 400 mL/min, about 300 mL/min to about 400 mL/min, about 100 mL/min to about 300 mL/min, about 200 mL/min to about 300 mL/min, or about 100 mL/min to about 200 mL/min. In certain embodiments, the blood flow rate is from about 100 mL/min to about 400 mL/min. In certain embodiments, the blood flow rate is from about 150 mL/min to about 250 mL/min. In certain embodiments, the blood flow rate is from about 135 mL/min to about 150 mL/min.

In certain embodiments, the ultrafiltration rate of the cartridge is from about 0 mL/min to about 180 mL/min, e.g., about 1 mL/min to about 180 mL/min. For example the ultrafiltration rate may be from about 1 mL/min to about 180 mL/min, about 5 mL/min to about 180 mL/min, about 20 mL/min to about 180 mL/min, about 40 mL/min to about 180 mL/min, about 60 mL/min to about 180 mL/min, about 80 mL/min to about 180 mL/min, about 100 mL/min to about 180 mL/min, about 120 mL/min to about 180 mL/min, about 140 mL/min to about 180 mL/min, about 160 mL/min to about 180 mL/min, about 1 mL/min to about 160 mL/min, about 5 mL/min to about 160 mL/min, about 20 mL/min to about 160 mL/min, about 40 mL/min to about 160 mL/min, about 60 mL/min to about 160 mL/min, about 80 mL/min to about 160 mL/min, about 100 mL/min to about 160 mL/min, about 120 mL/min to about 160 mL/min, about 140 mL/min to about 160 mL/min, about 1 mL/min to about 140 mL/min, about 5 mL/min to about 140 mL/min, about 20 mL/min to about 140 mL/min, about 40 mL/min to about 140 mL/min, about 60 mL/min to about 140 mL/min, about 80 mL/min to about 140 mL/min, about 100 mL/min to about 140 mL/min, about 120 mL/min to about 140 mL/min, about 1 mL/min to about 120 mL/min, about 5 mL/min to about 120 mL/min, about 20 mL/min to about 120 mL/min, about 40 mL/min to about 120 mL/min, about 60 mL/min to about 120 mL/min, about 80 mL/min to about 120 mL/min, about 100 mL/min to about 120 mL/min, about 1 mL/min to about 100 mL/min, about 5 mL/min to about 100 mL/min, about 20 mL/min to about 100 mL/min, about 40 mL/min to about 100 mL/min, about 60 mL/min to about 100 mL/min, about 80 mL/min to about 100 mL/min, about 1 mL/min to about 80 mL/min, about 5 mL/min to about 80 mL/min, about 20 mL/min to about 80 mL/min, about 40 mL/min to about 80 mL/min, about 60 mL/min to about 80 mL/min, about 1 mL/min to about 60 mL/min, about 5 mL/min to about 60 mL/min, about 20 mL/min to about 60 mL/min, about 40 mL/min to about 60 mL/min, about 1 mL/min to about 40 mL/min, about 5 mL/min to about 40 mL/min, about 20 mL/min to about 40 mL/min, about 1 mL/min to about 20 mL/min, about 5 mL/min to about 20 mL/min, or about 1 mL/min to about 5 mL/min. In certain embodiments, the ultrafiltration rate of the cartridge is from about 40 mL/min to about 180 mL/min.

In certain embodiments, the subject's blood is passed through the cartridge or contacted with the membrane before the subject receives a dose of chemotherapy or CAR T-cell therapy. In certain embodiments, the subject's blood is passed through the cartridge or contacted with the membrane simultaneously to the subject receiving a dose of chemotherapy or CAR T-cell therapy. In certain embodiments, the subject's blood is passed through the cartridge or contacted with the membrane after the subject receives a dose of chemotherapy or CAR T-cell therapy. In certain embodiments, the subject's blood is passed through the cartridge or contacted with the membrane simultaneously to and after the subject receives a dose of chemotherapy or CAR T-cell therapy.

In certain embodiments, the subject's blood is passed through the cartridge or contacted with the membrane for from about 0.5 to about 24 hours. For example, the subject's blood may be passed through the cartridge from about 0.5 to about 24, from about 0.5 to about 12, from about 0.5 to about 6, from about 0.5 to about 3, from about 0.5 to about 1, from about 1 to about 24, from about 1 to about 12, from about 1 to about 6, from about 1 to about 3, from about 3 to about 24, from about 3 to about 12, from about 3 to about 6, from about 6 to about 24, from about 6 to about 12, or from about 12 to about 24 hours. In certain embodiments, the subject's blood is passed through the cartridge or contacted with the membrane for about 1 hour, about 1 to about 3 hours, about 3 to about 6 hours, or about 6 to about 12 hours. In certain embodiments, the subject's blood is passed through the cartridge or contacted with the membrane for about 1 hour, about 3 hours, about 6 hours, about 9 hours, or about 12 hours.

In certain embodiments, the subject's blood is passed through the cartridge or contacted with the membrane within 0 to about 120 hours after the subject receives a dose of the chemotherapy or CAR T-cell therapy. For example, the subject's blood may be passed through the cartridge within from 0 to about 120, from 0 to about 96, from 0 to about 72, from 0 to about 48, from 0 to about 24, from 0 to about 12, from 0 to about 6, from 0 to about 3, from 0 to about 1, from about 0.5 to about 120, from about 0.5 to about 96, from about 0.5 to about 72, from about 0.5 to about 48, from about 0.5 to about 24, from about 0.5 to about 12, from about 0.5 to about 6, from about 0.5 to about 3, from about 0.5 to about 1, from about 1 to about 120, from about 1 to about 96, from about 1 to about 72, from about 1 to about 48, from about 1 to about 24, from about 1 to about 12, from about 1 to about 6, from about 1 to about 3, from about 3 to about 120, from about 3 to about 96, from about 3 to about 72, from about 3 to about 48, from about 3 to about 24, from about 3 to about 12, from about 3 to about 6, from about 6 to about 120, from about 6 to about 96, from about 6 to about 72, from about 6 to about 48, from about 6 to about 24, from about 6 to about 12, from about 12 to about 120, from about 12 to about 96, from about 12 to about 72, from about 12 to about 48, from about 12 to about 24, from about 24 to about 120, from about 24 to about 96, from about 24 to about 72, from about 24 to about 48, from about 48 to about 120, from about 48 to about 96, from about 48 to about 72, from about 72 to about 120, from about 72 to about 96, or from about 96 to about 120 hours after the subject receives a dose of the chemotherapy or CAR T-cell therapy. In certain embodiments, the method is performed on the subject prior to the subject exhibiting any symptoms of CRS.

In certain embodiments, the subject's blood is passed through the cartridge or contacted with the membrane within about 12 hours, about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks after the subject receives a dose of the chemotherapy or CAR T-cell therapy. In certain embodiments, the subject's blood is passed through the cartridge within 30 days, within 29 days, within 28 days, within 21 days, within 14 days, within 7 days, within 6 days, within 5 days, within 4 days, within 3 days, within 2 days or within 1 day after the subject receives a dose of the chemotherapy or CAR T-cell therapy.

In certain embodiments, the subject's blood is passed through the cartridge or contacted with the membrane subsequent to the subject receiving a dose of chemotherapy or CAR T-cell therapy more than once, for example, 1, 2, 3, 4, 5, 6, 7, 8, 9, 10, 11, 12, 14, 16, 18, 20, 22, 24, or more than 24 times. It is understood that an individual cartridge or membrane may be therapeutically effective for a limited amount of time, and as such, after that amount of time an individual cartridge or membrane will have to be replaced. Accordingly, when a subject's blood is passed through a cartridge or contacted with a membrane more than once, it is contemplated that in each instance the cartridge or membrane may be the same cartridge or membrane or a different cartridge or membrane.

In certain embodiments, the subject's blood is passed through the cartridge or contacted with the membrane multiples times (e.g., repeatedly, e.g., at regular intervals) over a treatment period subsequent to the subject receiving a dose of chemotherapy or CAR T-cell therapy. For example, in certain embodiments the subject's blood is passed through the cartridge or contacted with the membrane about every 12 hours, about every 24 hours, about every 2 days, about every 3 days, about every 4 days, about every 5 days, about every 6 days or about every 7 days, in each instance for about 1 hour, about 2 hours, about 3 hours, about 4 hours, about 5 hours, about 6 hours, about 9 hours, or about 12 hours, about every 3 hours, about every 6 hours, over a treatment period of about 1 day, about 2 days, about 3 days, about 4 days, about 5 days, about 6 days, about 1 week, about 2 weeks, about 3 weeks, about 4 weeks, about 6 weeks, about 8 weeks, about 10 weeks, or about 12 weeks.

In certain embodiments the subject's blood is passed through the cartridge or contacted with the membrane subsequent to the subject receiving a dose of chemotherapy or CAR T-cell therapy from about every day to about every 7 days, from about every day to about every 6 days, from about every day to about every 5 days, from about every day to about every 4 days, from about every day to about every 3 days, from about every day to about every 2 days, from about every 2 days to about every 7 days, from about every 2 days to about every 6 days, from about every 2 days to about every 5 days, from about every 2 days to about every 4 days, from about every 2 days to about every 3 days, from about every 3 days to about every 7 days, from about every 3 days to about every 6 days, from about every 3 days to about every 5 days, from about every 3 days to about every 4 days, from about every 4 days to about every 7 days, from about every 4 days to about every 6 days, from about every 4 days to about every 5 days, from about every 5 days to about every 7 days, from about every 5 days to about every 6 days, or from about every 6 days to about every 7 days. In certain embodiments, in each instance the subject's blood is passed through the cartridge or contacted with the membrane for from about 1 hour to about 2 hours, from about 1 hour to about 3 hours, from about 1 hour to about 4 hours, from about 1 hour to about 5 hours, from about 1 hour to about 6 hours, from about 1 hour to about 9 hours, from about 1 hour to about 12 hours, from about 2 hours to about 3 hours, from about 2 hours to about 4 hours, from about 2 hours to about 5 hours, from about 2 hours to about 6 hours, from about 2 hours to about 9 hours, from about 2 hours to about 12 hours from about 3 hours to about 4 hours, from about 3 hours to about 5 hours, from about 3 hours to about 6 hours, from about 3 hours to about 9 hours, from about 3 hours to about 12 hours, from about 4 hours to about 5 hours, from about 4 hours to about 6 hours, from about 4 hours to about 9 hours, from about 4 hours to about 12 hours, from about 5 hours to about 6 hours, from about 5 hours to about 9 hours, from about 5 hours to about 12 hours, from about 6 hours to about 9 hours, from about 6 hours to about 12 hours, or from about 9 hours to about 12 hours. In certain embodiments, the subject's blood is passed through the cartridge or contacted over a treatment period of from about 1 day to about 12 weeks, from about 1 day to about 10 weeks, from about 1 days to about 8 weeks, from about 1 day to about 6 weeks, from about 1 day to about 4 weeks, from about 1 day to about 2 weeks, from about 1 day to about 1 week, from about 1 day to about 5 days, from about 1 day to about 3 days, from about 3 days to about 12 weeks, from about 3 days to about 10 weeks, from about 3 days to about 8 weeks, from about 3 days to about 6 weeks, from about 3 days to about 4 weeks, from about 3 days to about 2 weeks, from about 3 days to about 1 week, from about 3 days to about 5 days, from about 5 days to about 12 weeks, from about 5 days to about 10 weeks, from about 5 days to about 8 weeks, from about 5 days to about 6 weeks, from about 5 days to about 4 weeks, from about 5 days to about 2 weeks, from about 5 days to about 1 week, from about 1 week to about 12 weeks, from about 1 week to about 10 weeks, from about 1 week to about 8 weeks, from about 1 week to about 6 weeks, from about 1 week to about 4 weeks, from about 1 week to about 2 weeks, from about 2 weeks to about 12 weeks, from about 2 weeks to about 10 weeks, from about 2 weeks to about 8 weeks, from about 2 weeks to about 6 weeks, from about 2 weeks to about 4 weeks, from about 4 weeks to about 12 weeks, from about 4 weeks to about 10 weeks, from about 4 weeks to about 8 weeks, from about 4 weeks to about 6 weeks, from about 6 weeks to about 12 weeks, from about 6 weeks to about 10 weeks, from about 6 weeks to about 8 weeks, from about 8 weeks to about 12 weeks, from about 8 weeks to about 10 weeks, or from about 10 weeks to about 12 weeks.

The composition of the material making up the blood pump, ultrafiltrate pump, IV fluid return pump, or tubing is preferably a biocompatible material, for example, polyvinylchloride. The tubing may be flexible and have dimensions complementary with associated hemofilter connections, ultrafiltrate recycling device connections, replacement fluid reservoir connection, joints, stop cocks, or pump heads.

In certain embodiments, fluid circuits incorporating the membrane or cartridge optionally can also perform other blood treatments. For example, fluid circuits optionally can further include additional devices that can filter, oxygenate, warm, or otherwise treat the blood before or after the blood enters the cartridge.

In certain embodiments, the membranes, cartridges and/or the fluid circuits incorporating the membranes or cartridges are controlled by a processor (e.g., computer software). In such embodiments, a device can be configured to detect changes within a subject and provide such information to the processor. In some embodiments, the fluid circuit can automatically process the subject's blood through the cartridge in response to such information. In other embodiments, a health professional is alerted and initiates treatment.

Exemplary membranes, cartridges, and blood circuits that may be useful in the practice of the invention are disclosed, for example, in U.S. Pat. Nos. 8,597,516 and 6,787,040.

### III. Therapeutic Uses

The methods disclosed herein can be used to improves outcomes in a subject with or at risk for Cytokine Release Syndrome (CRS) or Tumor Lysis Syndrome (TLS), for example, a subject undergoing CAR T-cell therapy or chemotherapy.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, primates (e.g., simians), equines, bovines, porcines, canines, felines, and the like). In a preferred embodiment, the subject is a human subject, including both adult and pediatric human subjects.

The term “effective amount” as used herein refers to the amount of a method or agent sufficient to effect beneficial or desired results. An effective amount can be administered in one or more administrations, applications or dosages and is not intended to be limited to a particular formulation or administration route.

The invention provides a method of treating, preventing, or reducing the risk of CRS or TLS in a subject, e.g., a subject undergoing CAR T-cell therapy or chemotherapy. The invention also provides a method of increasing drug exposure in a subject, e.g., a subject undergoing CAR T-cell therapy or chemotherapy, while minimizing the risk that the subject experiences CRS or TLS.

As used herein, the terms “chimeric antigen receptor,” or “CAR,” refer to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T-cell signaling molecules (e.g. a T-cell costimulatory domain (e.g., from CD28, CD137, OX40, ICOS, or CD27) in tandem with a T-cell triggering domain (e.g. from CD3)) and/or to cytoplasmic domains of NK-cell signaling molecules (e.g. DNAX-activation protein 12 (DAP12)).

It is contemplated that a CAR can be expressed in any immune cell. Immune cells include, e.g., lymphocytes, such as B-cells and T-cells, natural killer cells, myeloid cells, such as monocytes, macrophages, eosinophils, mast cells, basophils, and granulocytes. In certain embodiments, the immune cell is a T-cell, which can be, for example, a cultured T-cell, e.g., a primary T-cell, or a T-cell from a cultured T-cell line, e.g., Jurkat, SupTi, etc., or a T-cell obtained from a mammal, for example, from a subject to be treated. If obtained from a mammal, the T-cell can be obtained from numerous sources, including but not limited to blood, bone marrow, lymph node, the thymus, or other tissues or fluids. T-cells can also be enriched or purified. The T-cell can be any type of T-cell and can be of any developmental stage, including but not limited to, CD4+/CD8+ double positive T-cells, CD4+ helper T-cells, e.g., Th1 and Th2 cells, CD4+ T-cells, CD8+ T-cells (e.g., cytotoxic T-cells), tumor infiltrating lymphocytes (TILs), memory T-cells (e.g., central memory T-cells and effector memory T-cells), naive T-cells, and the like. The cells (e.g., the T-cells) can include autologous cells derived from a subject to be treated, or alternatively allogenic cells derived from a donor. A T-cell expressing or otherwise including a chimeric antigen receptor is referred to as a CAR T-cell and an NK-cell expressing or otherwise including a chimeric antigen receptor is referred to as a CAR NK-cell. In one embodiment the CAR is expressed in a T-cell, i.e., a CAR T-cell.

Exemplary CAR T-cells include KYMRIAH® (Tisagenlecleucel) or YESCARTA® (Axicabtagene ciloleucel). Additional exemplary CAR T-cells are described in U.S. Pat. Nos. 8,399,645, 8,906,682, 7,446,190, 9,181,527, 9,272,002, and 9,266,960, U.S. Patent Publication Nos. US20160362472, US20160200824, and US20160311917 and International (PCT) Publication Nos. WO2013142034, WO2015188141, WO2017040945, WO2015120180, and WO2016120220.

It is contemplated that the methods described herein allow for administration of a CAR T-cell therapy at a higher dose than would otherwise be possible, for example, at a dose that is higher than the dose of the CAR T-cell therapy that is approved for use in human subjects by the U.S. Food and Drug Administration (FDA) or the European Medicines Agency (EMA). In certain embodiments, the methods prevent the need for dose-reduction of CAR T-cell therapy to prevent CRS or TLS. For example, in certain embodiments, the methods described herein allow for administration of KYMRIAH® (Tisagenlecleucel) at a dose that is (i) greater than 5.0×10⁶ CAR-positive viable T cells per kg weight intravenously and the subject is a pediatric or young adult subject up to 25 years of age with B-cell acute lymphoblastic leukemia weighing 50 kg or less, (ii) greater than 2.5×10⁸ CAR-positive viable T cells intravenously and the subject is a pediatric or young adult subject with B-cell acute lymphoblastic leukemia weighing 50 kg or more, or (iii) greater than 6.0×10⁸ CAR-positive viable T cells and the subject is an adult subject with relapsed or refractory diffuse large B-cell lymphoma. In certain embodiments, the methods described herein allow for administration of YESCARTA® (Axicabtagene ciloleucel) at a dose that is (i) greater than 2×10⁶ CAR-positive viable T cells per kg body weight, or (ii) greater than 2×10⁸ CAR-positive viable T cells.

As used herein, “chemotherapy” refers to chemical agents used to treat cancer. Chemotherapy includes small molecule drugs, protein based drugs, and metallic complexes. In certain embodiments, the chemotherapy may include an antibody. As used herein, unless otherwise indicated, the term “antibody” is understood to mean an intact antibody (e.g., an intact monoclonal antibody), or a fragment thereof, such an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

Exemplary antibodies include ado-trastuzumab emtansine, alemtuzumab, avelumab, atezolizumab, belantamab mafodotin, bevacizumab, brentuximab vedotin, cemiplimab, cetuximab, dacetuzumab, daratumumab, dinutuximab, dostarlimab, durvalumab, edrecolomab, elotuzumab, enfortumab vedotin, fam-trastuzumab deruxtecan, gemtuzumab ozogamicin, ibritumomab tiuxetan, inotuzumab ozogamicin, ipilimumab, isatuximab, margetuximab, mogamuizumab, moxetumomab pasudotox, naxitamab, necitumumab, nivolumab, obinutuzumab, ofatumumab, olaratumab, oportuzumab monatox, panitumumab, pembrolizumab, pertuzumab, polatuzumab vedotin, ramucirumab, rituximab, sacituzumab govitecan, tafasitamab, theralizumab (also known as TGN1412), trastuzumab, and tositumomab-I131. Exemplary bispecific antibodies include blinatumomab and catumaxomab.

In certain embodiments, the chemotherapy may include a cytotoxic agent. Exemplary cytotoxic agents include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In certain embodiments, the cytotoxic agent is a platinum-based agent (such as cisplatin or oxaliplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, lenalidomide, or maytansinoids.

In certain embodiments the chemotherapy may include may include a targeted therapy, e.g. a tyrosine kinase inhibitor, a proteasome inhibitor, or a protease inhibitor. In certain embodiments, the chemotherapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the chemotherapy may include may include a combination of therapeutics of different classes.

It is contemplated that the methods described herein allow for administration of a chemotherapy at a higher dose than would otherwise be possible, for example, at a dose that is higher than the dose of the chemotherapy that is approved for use in human subjects by the U.S. Food and Drug Administration (FDA) or the European Medicines Agency (EMA). In certain embodiments, the methods prevent the need for dose-reduction of the chemotherapy to prevent CRS or TLS.

In certain embodiments, the methods disclosed herein remove inflammatory molecules from the blood of a subject. The removal can occur before, during, or after the subject receives a dose of chemotherapy or CAR T-cell therapy. The methods described herein may reduce systemic inflammation in a subject. For example, methods and cartridges described herein may reduce a level of an inflammatory molecule in a recipient, e.g., in a body fluid (e.g., blood, plasma, serum, or urine), tissue and/or cell in a recipient, e.g., by at least about 1%, about 5%, about 10%, about 20%, about 30%, about 40%, about 50%, about 60%, about 70%, about 80%, about 90%, about 95%, or more, relative to levels in an untreated or control subject. For example, methods and cartridges described herein may reduce a level of an inflammatory cytokine or chemokine. Exemplary inflammatory cytokines or chemokines include IL-1-β, IL-4, IL-5, IL-6, IL-8, IL-12, IL-13, IL-17, IL-21, IL-22, IL-23, IL-27, IFN, CCL-2, CCL-3, CCL-5, CCL-20, CXCL-5, CXCL-10, CXCL-12, CXCL-13, and TNF-α. Additional exemplary inflammatory molecules include MCP-1, IP-10, C3a, C5a, soluble TNF receptor II, soluble TNF receptor I, matrix metalloproteinase-9, matrix metalloproteinase-7, IL-10, soluble gp130, lipopolysaccharide, and procalcitonin. In certain embodiments, the inflammatory cytokines or chemokines that may be removed from the subject can include one or more of IL-6, TNF-α, C3a, and C5a. It is understood that reference to an inflammatory molecule includes the inflammatory molecule in both an unbound state or in complex with a corresponding ligand. Exemplary inflammatory molecule-ligand complexes include an IL-6/IL-6 soluble receptor complex, a TNF-α/soluble TNF receptor complex, and an albumin/inflammatory cytokine complex.

In certain embodiments, the methods disclosed herein remove one or more metabolites resulting from tumor cell lysis. The removal can occur before, during, or after the subject receives a dose of chemotherapy or CAR T-cell therapy. For example, methods and cartridges described herein may reduce a level of a metabolite in a subject, e.g., in a body fluid (e.g., blood, plasma, serum, or urine), tissue and/or cell in a subject, e.g., by at least about 1%, about 5%, about 10%, about 20%, about 30%, about 40%, about 50%, about 60%, about 70%, about 80%, about 90%, about 95%, or more, relative to levels in an untreated or control subject. Exemplary metabolites resulting from tumor cell lysis include uric acid, potassium and phosphorus. For example, in certain embodiments, (i) the method maintains the level of uric acid in the subject at or below 476 μmol/L, (ii) the method maintains the level of potassium in the subject at or below 6 mmol/L, and/or (iii) the method maintains the level of phosphorus in the subject at or below 1.45 mmol/L.

In certain embodiments, a subject treated with a method of the invention has cancer. Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma). In certain embodiments, the cancer is a hematologic cancer, e.g., a leukemia, lymphoma, or multiple myeloma, e.g., chronic lymphocytic leukemia (CLL), acute myeloid leukemia (AML), chronic myelogenous leukemia (CIVIL), acute lymphoblastic B cell leukemia (B-ALL), diffuse large B cell lymphoma (DLBCL), non-Hodgkin lymphoma (NHL), Burkitt lymphoma, chronic myeloid monocytic leukemia (CMML), eosinophilia, essential thrombocytosis, hairy cell leukemia, and NK cell lymphoma.

The methods described herein can be used alone or in combination with other therapeutic agents and/or modalities. The term administered “in combination,” as used herein, is understood to mean that two (or more) different treatments are delivered to the subject, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment improves an outcome to a greater extent, than would be seen if the second treatment were administered in the absence of the first treatment, or the analogous situation is seen with the first treatment. In certain embodiments, delivery is such that improvement of an outcome is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a disclosed method is administered in combination with albumin, e.g., the subject receives exchange of recombinant or pharmaceutical grade albumin.

In certain embodiments, a disclosed method is administered in combination with an anticoagulant, for example, heparin, a citrate salt, etc. Anticoagulation protocols, such as systemic heparin or regional citrate, are currently established and routinely used in clinical hemofiltration. Additional exemplary anticoagulants include warfarin, FXa inhibitors (e.g., enoxaparin, rivaroxaban, apixaban, betrixaban and edoxaban), thrombin inhibitors (e.g., hirudin, lepirudin, bivalirudin, argatroban and dabigatran), and coumarins.

In certain embodiments, the subject does not receive tocilizumab prior to or subsequent to administration of a dose of chemotherapy or CAR-T therapy.

In certain embodiments, the methods disclosed herein reduce the risk of a subject developing sepsis from concomitant infection with a bacteria, virus or fungus. In certain embodiments, the methods negate the need for the subject to receive allopurinol or a phosphate binder. In certain embodiments, the methods negate the need for the subject to receive corticosteroids.

Throughout the description, where devices or compositions are described as having, including, or comprising specific components, or where processes and methods are described as having, including, or comprising specific steps, it is contemplated that, additionally, there are devices or compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a device, a composition, or a method described herein can be combined in a variety of ways without departing from the spirit and scope of the present invention, whether explicit or implicit herein. For example, where reference is made to a particular device, that device can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remains operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes the prevention of Cytokine Release Syndrome (CRS) in a subject undergoing CAR T-cell therapy with a hemofilter.

Subjects are treated for 6 to 12 hours after treatment with CAR T-cell therapy or chemotherapy with a CLR 2.0 Hemofilter (Seastar Medical, Inc., Denver, Colo.). The filtrate-substitution fluid exchange dose is 35 mL/kg/hour. Substitution fluid is delivered pre-dilution (upstream of the hemofilter). Fluid removal is as indicated by the subject's hydration status. Vascular access is, for example, by a size 13.5 to 14.5 French scale (Fr) dialysis catheter in the right internal jugular vein, left internal jugular vein, and either femoral vein. Subjects receive no anticoagulation therapy.

It is expected that treatment reduces the risk of developing CRS as a result of the CAR T-cell therapy. It is also expected that treatment reduces or eliminates the need for the subject to receive allopurinol, a phosphate binder, or a corticosteroid. Additionally, reduced risk of developing CRS allows for a higher dose of the CAR T-cell therapy and/or prevents the need for dose reduction.

Together, these results demonstrate that treatment of a subject undergoing CAR T-cell therapy with a hemofilter improves outcomes.

### Example 2

This Example describes the prevention of Tumor Lysis Syndrome (TLS) in a subject undergoing CAR T-cell therapy with a hemofilter.

Subjects are treated for 6 to 12 hours after treatment with CAR T-cell therapy or chemotherapy with a CLR 2.0 Hemofilter (Seastar Medical, Inc., Denver, Colo.). The filtrate-substitution fluid exchange dose is 35 mL/kg/hour. Substitution fluid is delivered pre-dilution (upstream of the hemofilter). Fluid removal is as indicated by the subject's hydration status. Vascular access is, for example, by a size 13.5 to 14.5 French scale (Fr) dialysis catheter in the right internal jugular vein, left internal jugular vein, and either femoral vein. Subjects receive no anticoagulation therapy.

It is expected that treatment reduces the risk of developing TLS as a result of the CAR T-cell therapy. It is expected that treatment (i) maintains the level of uric acid in the subject at or below 476 μmol/L, (ii) maintains the level of potassium in the subject at or below 6 mmol/L, and/or (iii) maintains the level of phosphorus in the subject at or below 1.45 mmol/L. It is also expected that treatment reduces or eliminates the need for the subject to receive allopurinol, a phosphate binder, or a corticosteroid. Additionally, reduced risk of developing TLS allows for a higher dose of the CAR T-cell therapy or chemotherapy and/or prevents the need for dose reduction.

Together, these results demonstrate that treatment of a subject undergoing CAR T-cell therapy with a hemofilter improves outcomes.

### Example 3

This Example describes the treatment of Cytokine Release Syndrome (CRS) in a subject undergoing CAR T-cell therapy with a hemofilter.

A subject receiving CAR T-cell therapy and showing symptoms of CRS is connected to a CLR 2.0 Hemofilter (Seastar Medical, Inc., Denver, Colo.) as described herein. The filtrate-substitution fluid exchange dose is 35 mL/kg/hour. Substitution fluid is delivered pre-dilution (upstream of the hemofilter). Fluid removal is as indicated by the subject's hydration status. Vascular access is, for example, by a size 13.5 to 14.5 French scale (Fr) dialysis catheter in the right internal jugular vein, left internal jugular vein, and either femoral vein. The subject receives no anticoagulation therapy. The subject remains connected to the device for 6-12 hours, after which treatment the subject's symptoms of CRS subside.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of reducing the risk of Cytokine Release Syndrome (CRS) in a subject undergoing CAR T-cell therapy or chemotherapy, the method comprising:
(i) contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient; or
(ii) passing blood from the recipient through an extracorporeal cartridge comprising a housing and a plurality of semi-permeable hollow fibers disposed therein, each of the semi-permeable hollow fibers defining a lumen and a plurality of pores having an average pore size of at least 60 kDa, such that when the blood traverses the lumens of the hollow fibers, inflammatory molecules from the blood pass through the pores and are removed from the blood, whereupon the blood depleted of inflammatory molecules is returned to the recipient.

2. A method of increasing exposure to CAR T-cell therapy or chemotherapy in a subject undergoing CAR T-cell therapy or chemotherapy while minimizing the risk that the subject experiences Cytokine Release Syndrome (CRS), the method comprising:
(i) contacting blood from the recipient with an extracorporeal membrane defining a plurality of pores having an average pore size of at least 60 kDa to permit inflammatory molecules in the blood to pass through the pores for removal from the blood, whereupon the blood depleted of the inflammatory molecules is returned to the recipient; or
(ii) passing blood from the recipient through an extracorporeal cartridge comprising a housing and a plurality of semi-permeable hollow fibers disposed therein, each of the semi-permeable hollow fibers defining a lumen and a plurality of pores having an average pore size of at least 60 kDa, such that when the blood traverses the lumens of the hollow fibers, inflammatory molecules from the blood pass through the pores and are removed from the blood, whereupon the blood depleted of inflammatory molecules is returned to the recipient.

3. A method of reducing the risk of Tumor Lysis Syndrome (TLS) in a subject undergoing CAR T-cell therapy or chemotherapy, the method comprising:
(i) contacting blood from the recipient with an extracorporeal membrane defining a plurality of pores having an average pore size of at least 60 kDa to permit inflammatory molecules and metabolites resulting from tumor lysis in the blood to pass through the pores for removal from the blood, whereupon the blood depleted of the inflammatory molecules and metabolites resulting from tumor cell lysis is returned to the recipient; or
(ii) passing blood from the recipient through an extracorporeal cartridge comprising a housing and a plurality of semi-permeable hollow fibers disposed therein, each of the semi-permeable hollow fibers defining a lumen and a plurality of pores having an average pore size of at least 60 kDa, such that when the blood traverses the lumens of the hollow fibers, inflammatory molecules and metabolites resulting from tumor lysis in the blood pass through the pores and are removed from the blood, whereupon the blood depleted of inflammatory molecules and metabolites resulting from tumor cell lysis is returned to the recipient.

4. A method of increasing exposure to CAR T-cell therapy or chemotherapy in a subject undergoing CAR T-cell therapy or chemotherapy while minimizing the risk that the subject experiences Tumor Lysis Syndrome (TLS), the method comprising:
(i) contacting blood from the recipient with an extracorporeal membrane defining a plurality of pores having an average pore size of at least 60 kDa to permit inflammatory molecules and metabolites resulting from tumor lysis in the blood to pass through the pores for removal from the blood, whereupon the blood depleted of the inflammatory molecules and metabolites resulting from tumor lysis is returned to the recipient; or
(ii) passing blood from the recipient through an extracorporeal cartridge comprising a housing and a plurality of semi-permeable hollow fibers disposed therein, each of the semi-permeable hollow fibers defining a lumen and a plurality of pores, such that when the blood traverses the lumens of the hollow fibers, inflammatory molecules and metabolites resulting from tumor lysis in the blood pass through the pores and are removed from the blood, whereupon the blood depleted of inflammatory molecules and metabolites resulting from tumor cell lysis is returned to the recipient.

5. The method of claim 3 or 4, wherein the metabolites resulting from tumor cell lysis are one or more of uric acid, potassium and phosphorus.

6. The method of claims 3-5, wherein the method maintains the level of uric acid in the subject at or below 476 μmol/L.

7. The method of claims 3-5, wherein the method maintains the level of potassium in the subject at or below 6 mmol/L.

8. The method of claims 3-5, wherein the method maintains the level of phosphorus in the subject at or below 1.45 mmol/L.

9. The method of claims 3-8, wherein the subject also has a reduced risk of developing Cytokine Release Syndrome (CRS) as a result of the method.

10. The method of claims 1-9, wherein the method is performed on the subject within 0-24 hours after the subject receives a dose of the chemotherapy or CAR T-cell therapy.

11. The method of claims 1-9, wherein the method is performed on the subject within 0-72 hours after the subject receives a dose of the chemotherapy or CAR T-cell therapy.

12. The method of claims 1-11, wherein the method is performed on the subject prior to the subject exhibiting any symptoms of CRS.

13. The method of claims 1-12, wherein the subject does not receive tocilizumab prior to or subsequent to administration of a dose of chemotherapy or CAR T-cell therapy

14. The method of claims 1-13, wherein the chemotherapy is an antibody therapy.

15. The method of claims 1-14, wherein the chemotherapy is a bispecific antibody therapy.

16. The method of claims 1-15, wherein the chemotherapy is a non-protein based chemotherapeutic.

17. The method of claims 1-16, wherein the chemotherapeutic is anti-thymocyte globulin (ATG), TGN1412, rituximab, obinutuzumab, alemtuzumab, brentuximab, dacetuzumab, nivolumab, oxaliplatin, lenalidomide, or blinatolimumab.

18. The method of claims 1-13, wherein the CAR T-cell therapy is KYMRIAH® (Tisagenlecleucel) or YESCARTA® (Axicabtagene ciloleucel).

19. The method of claims 1-18, wherein the subject has a hematologic malignancy.

20. The method of claim 19, wherein the malignancy is acute lymphoblastic B cell leukemia (B-ALL), chronic lymphocytic leukemia (CLL), diffuse large B cell lymphoma (DLBCL), acute myeloid leukemia (AML), or Burkitt Lymphoma.

21. The method of claims 1-20, wherein the CAR T-cell therapy is administered at a dose greater than the U.S. Food and Drug Administration approved dose.

22. The method of claims 21, wherein the CAR T-cell therapy is KYMRIAH®.

23. The method of claim 22, wherein the dose of KYMRIAH® is greater than 5.0×10⁶ CAR-positive viable T cells per kg weight intravenously and the subject is a pediatric or young adult subject up to 25 years of age with B-cell acute lymphoblastic leukemia weighing 50 kg or less or the dose is greater than 2.5×10⁸ CAR-positive viable T cells intravenously and the subject is a pediatric or young adult subject with B-cell acute lymphoblastic leukemia weighing 50 kg or more.

24. The method of claim 22, wherein the dose of KYMRIAH® is greater than 6.0×10⁸ CAR-positive viable T cells and the subject is an adult subject with relapsed or refractory diffuse large B-cell lymphoma.

25. The method of claim 21, wherein the CAR T-cell therapy is YESCARTA®.

26. The method of claim 25, wherein the dose of YESCARTA® is greater than 2×10⁶ CAR-positive viable T cells per kg body weight or is greater than 2×10⁸ CAR-positive viable T cells.

27. The method of claims 1-26, wherein the method reduces the likelihood the subject will experience sepsis from concomitant infection with a bacteria, virus or fungus.

28. The method of claims 1-27, wherein the method prevents the need for dose-reduction of the chemotherapy or CAR T-cell therapy to prevent CRS.

29. The method of claims 3-16, wherein the method negates the need for the subject to receive allopurinol or a phosphate binder.

30. The method of claims 1-29, wherein the method negates the need for the subject to receive corticosteroids.

31. The method of any one of claims 1-30, wherein the pores are defined by a wall of a semi-permeable hollow fiber.

32. The method of any one of claims 1-31, wherein the pores have an average pore size of from about 60 kDa to about 150 kDa.

33. The method of any one of claims 1-31 wherein the pores have an average pore size of greater than 65 kDa.

34. The method of any one of claims 1-31, wherein the pores have an average pore size of no greater than 65 kDa.

35. The method of any one of claims 1-31, wherein the pores have an average pore size from about 60 kDa to about 65 kDa.

36. The method of any one of claims 1-35, wherein the subject receives exchange of recombinant or pharmaceutical grade albumin.

37. The method of any one of claims 1-36, wherein the subject is human.

38. The method of any one of claims 1-37, wherein the subject is an adult human.

39. The method of any one of claims 1-37, wherein the subject is a pediatric human.

40. The method of any one of claims 1-39, wherein the inflammatory molecules removed from the blood are selected from one or more of IL-4, IL-6, IL-8, TNF-α, IL-β, MCP-1, CCL2, IP-10, CXCL10, C3a, C5a, soluble TNF receptor II, soluble TNF receptor I, matrix metalloproteinase-9, matrix metalloproteinase-7, IL-10, soluble gp130, lipopolysaccharide (LPS), or procalcitonin.
